(19) United States
(12) Patent Application Publication
(10) Pub. No.: US 2004/0127742 A1
(43) Pub. Date: Jul. 1, 2004

(54) Alcohol ether sulfonates

(75) Inventors: Prakasa R. Anantaneni, Austin, TX (US); Samir S. Ashrawi, Austin, TX (US); David C. Lewis, Austin, TX (US); George A. Smith, Austin, TX (US)

Correspondence Address: Huntsman LLC, Legal Department, P.O. Box 15730, Austin, TX, 78761, US

(73) Assignee: Huntsman Petrochemical Corporation, Austin, TX

(21) Appl. No.: 10/690,467

(22) Filed: Oct. 21, 2003

Related U.S. Application Data
(60) Provisional application No. 60/420,238, filed on Oct. 22, 2002.
(60) Provisional application No. 60/420,362, filed on Oct. 22, 2002.

(51) Int. Cl. C 07C 3 9/10

(52) U.S. Cl. 562/110

(57) ABSTRACT

Provided herein is a class of compounds with surfactant properties known as alcohol ether sulfonates. These materials are produced according to the present invention by reaction of isethionic acid or its halo-derivative with an alcohol. An alcohol ether sulfonate according to the invention is useful as a component of a finished surfactant formulation useful for cleaning hard surfaces, laundry, and dishes, among other things.

### CROSS-REFERENCES TO RELATED APPLICATIONS

This application claims priority to U.S. Provisional Patent Application having serial No. 60/420,238 having filing date Oct. 22, 2002 and to to U.S. Provisional Patent Application having serial No. 60/420,362 also having filing date Oct. 22, 2002 both of which are currently still pending.

### FIELD OF THE INVENTION

This invention relates to compositions of matter useful as surfactants. More particularly it relates to a process for producing alcohol ether sulfonates from an isethionic acid or its derivative and a primary alcohol.

### BACKGROUND

The surfactant art is replete with compounds possessing surface active properties of various molecular structures. Generally, surfactants contain a hydrophilic and a hydrophobic portion (typically polar and non-polar, respectively) which may include molecules that carry a net electrical charge. Alkyloxyethane sulfonic acids and their corresponding salts are a subclass of materials which are well known to be useful as surfactants, and are especially useful in personal care applications, such as hand and body soaps. These materials are particularly well suited to be used at low pH levels, where conventional soaps tend to precipitate out of solution.

Currently, the sodium salt of alkyloxyethane sulfonates are made by reaction of sodium vinyl sulfonate or sodium isethionate using a base catalyst. The former appears in commercial use, while the latter is not, probably owing to difficulties in obtaining acceptable yields to make the process economical. The reaction of sodium vinyl sulfonate with a base catalyst is conducted at about 170° C.

We have discovered that materials having beneficial surfactant properties are produced from the reaction of an alcohol with isethionic acids, and alternatively from the reaction of an alcohol with a halogen-substituted isethionic acid to form ethers which we term “alcohol ether sulfonates”. Such materials we have found are relatively low in cost and effort to produce, and yield some of the same benefits as do alkane sulfonates, which are a class of materials that are much more difficult to produce.

### SUMMARY OF THE INVENTION

The present invention provides a process for producing an alcohol ether sulfonate by reacting an alcohol with isethionic acid or its halo-derivative according to the reaction:

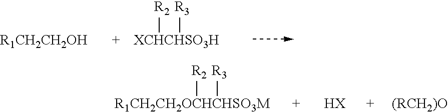

in which:

R₁ is a straight-chain, branched, or cyclic, saturated or unsaturated, hydrocarbyl moiety that is selected from the group consisting of: 1) any C₅-C₁₉ alkyl group; 2) any C₅-C₁₉ aryl group; 3) any C₅-C₁₉ alkylaryl group; 4) any R₄(CH₂CH₂O)ₙ— group, in which R₄ is any C₃-C₂₄ alkyl, aryl, or alkylaryl group, whether straight-chain, branched, or cyclic, saturated or unsaturated, and in which n independently has any value between about 2 and 25;

R₂ and R₃ are each independently selected from the group consisting of: hydrogen, methyl and ethyl;

X is selected from the group consisting of: chlorine, bromine, or hydroxy; and

M is selected from the group consisting of: Na, K, Li, Ca, Mg, and hydrogen, by contacting the isethionic acid or its halo-derivative and the primary alcohol at any temperature in the range of about 60° C. to about 200° C., and at any pressure in the range of between about 50 and 760 mm Hg.

The present invention also concerns compositions of matter comprising such ether isethionates in combination with other materials known to those skilled in the art of soaps, detergents, and the like to produce finished formulations useful as cleaning compositions.

### DETAILED DESCRIPTION

The present invention concerns a process for producing an alcohol ether sulfonate by reacting an alcohol with isethionic acid according to the reaction:

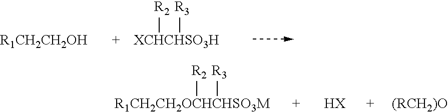

in which:

R₁ is independently any straight-chain, branched, or cyclic, saturated or unsaturated, hydrocarbyl moiety that is selected from the group consisting of: 1) any C₅-C₁₉ alkyl group; 2) any C₅-C₁₉ aryl group; 3) any C₅-C₁₉ alkylaryl group; 4) any R₄(CH₂CH₂O)ₙ— group, in which R₄ is any C₃-C₂₄ alkyl, aryl, or alkylaryl group, whether straight-chain, branched, or cyclic, saturated or unsaturated, and in which n independently has any value between about 2 and 25;

R₂ and R₃ are each independently selected from the group consisting of: hydrogen, methyl and ethyl;

X is selected from the group consisting of: chlorine, bromine, or hydroxy; and

M is selected from the group consisting of: Na, K, Li, Ca, Mg, and hydrogen, by contacting the isethionic acid or its halo-derivative and the primary alcohol at any temperature in the range of about 60° C. to about 200° C., and at any pressure in the range of between about 50 and 760 mm Hg.

The invention is also concerned with formulations which contain such ether isethionates which are useful as cleaning agents.

To carry out a reaction according to the present invention, one begins by charging the alcohol and the isethionic acid or its halogenated derivative to a reaction vessel. The contents are then heated, preferably to a temperature in the range of between 60° C. and 200° C. and for an amount of time between about 4 and 20 hours. According to a preferred process according to the invention, the isethionic acid or its derivative is added slowly to a C₈-C₁₈ alcohol (or mixture of alcohols in this carbon range) that is maintained at any temperature in the range of between about 100° to 130° C. As the reaction proceeds, water is formed from the condensation. Preferably, the reaction apparatus includes a Dean-Stark or similar type trap to remove the water as it is formed and to thus drive the reaction towards completion. The removal of water is facilitated either by sweeping the reactor overhead with nitrogen, or by the application of a slight vacuum in the range of about 100 to about 700 mm below atmospheric pressure. It is generally preferred to keep the reaction temperature as low as possible while maintaining a reasonable reaction rate, so as to avoid the formation of color bodies in the final product resulting from overheating.

According to a preferred form of the invention, an excess of alcohol reactant is employed, which also conveniently serves as a solution in which the reaction occurs. Thus, for each mole of isethionic acid or isethionic acid derivative employed, it is preferred to have present an amount of alcohol present that is equal to between about 1.5 and about 4 moles of the alcohol in order to optimize the amount of alcohol ether sulfonate present in the reaction mixture when the reaction has equilibrated. If it is desirable to maximize the yield of dialkyl ether present, then ratios of the amount of alcohol used to isethionic acid derivative present is greater than 4, and is preferably in the range of between about 4:1 to about 8:1.

The final product alcohol ether sulfonate may be separated from ether co-product, which is invariably formed to some extent more or less attendant with the alcohol ether sulfonate, by adding aqueous alkali to the reaction product mixture so as to cause the alcohol ether sulfonate to exist in its anionic form in an aqueous phase, and causing the ether co-product to exist in the organic phase. Separation of the two phases using conventional techniques affords an aqueous layer comprising only the alcohol ether sulfonate, which may be re-acidified if desired to yield the acid form, or left in anionic form and the solution used as-is or further processed.

To make a final product blend comprising an alcohol ether sulfonate of the present invention, one merely mixes the various components using conventional blending techniques. Heat may be applied in those cases where it is desired to make blends containing materials which are solids at room temperature.

Other components known to those skilled in the art of formulating soaps, detergents, and the like, which may be combined with an alcohol ether sulfonate according to the present invention include without limitation: fatty acids, alkyl sulfates, ethanolamines, amine oxides, alkali carbonates, water, ethanol, isopropanol, pine oil, sodium chloride, sodium silicate, polymers, alcohol alkoxylates, zeolites, perborate salts, alkali sulfates, enzymes, hydrotropes, dyes, fragrances, preservatives, brighteners, builders, polyacrylates, essential oils, alkali hydroxides, ether sulfates, alkylphenol ethoxylates, fatty acid amides, alpha olefin sulfonates, paraffin sulfonates, betaines, chelating agents, tallowamine ethoxylates, polyetheramine ethoxylates, ethylene oxide/propylene oxide block copolymers, alcohol ethylene oxide/propylene oxide low foam surfactants, methyl ester sulfonates, alkyl polysaccharides, N-methyl glucamides, alkylated sulfonated diphenyl oxide, and water soluble alkylbenzene sulfonates or alkyltoluene sulfonates, regardless of their 2-phenyl isomer content or degree of branching or linearity in the alkyl chain.

A finished composition according to one form of the invention comprises an anionic form of the alcohol ether sulfonate described by the formula:

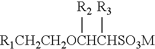

in which R₁ is independently any straight-chain, branched, or cyclic, saturated or unsaturated, hydrocarbyl moiety that is selected from the group consisting of: 1) any C₅-C₁₉ alkyl group; 2) any C₅-C₁₉ aryl group; 3) any C₅-C₁₉ alkylaryl group; 4) any R₄(CH₂CH₂O)ₙ— group, in which R₄ is any C₃-C₂₄ alkyl, aryl, or alkylaryl group, whether straight-chain, branched, or cyclic, saturated or unsaturated, and in which n independently has any value between about 2 and 25; R₂ and R₃ are each independently selected from the group consisting of: hydrogen, methyl and ethyl; and M is selected from the group consisting of: Na, K, Li, Ca, Mg, and hydrogen. An alcohol ether sulfonate according to the invention will invariably be present in its anionic form in the case where the solution of medium in which such material is contained is acidic, because these materials are “strong” acids and are essentially completely dissociated in water. In the case where the acidity has been neutralized, a counter-ion will be present to provide electrical neutrality to the materials. Such counter-ions include without limitation such ions as sodium, potassium, ammonium, substituted ammonium, rubidium, cesium, magnesium, calcium, strontium, aluminum, etc.

In addition to their use as being components of a formulation as described above, the alcohol ether sulfonates of the present invention are anticipated as finding utility as: hydrotropes, oilfield surfactants, caustic cleaners for CIP, hard surface cleaners, and textile adjuvants.

The process to manufacture ethane sulfonates or propane sulfonates of the prior art is quite involved. In our process a simple one step reaction is used and produced product with light color, in the acid form and without washing steps. The strong acidity of the isethionic acid has been found to make the elimination easy. Products produced by this process are expected to be very stable compounds, with excellent hydrolytic stability, caustic solubility, and high temperature stability.

### EXAMPLE 1

A four-necked 500 ml round-bottom flask equipped with a mechanical stirrer, an addition funnel, nitrogen inlet, and a thermocouple for temperature control, is charged with 200 g (1.0 mole) C₁₂-C₁₄ alcohol and heated to about 125°-130° C. 160 grams of an 80% (wt. %) aqueous solution of isethionic acid is added slowly over 45-60 minutes with stirring. Water from ISA is removed by vaporization as it is added and after the addition the temperature is raised slowly to 160°-165° C. over 30 minutes. The ISA layer turns dark as the temperature is increased but more water came over than anticipated. The total mixture is neutralized with about 80 grams of an aqueous solution comprising 50% sodium hydroxide, and subsequently diluted with 150 g of water. The organic layer was separated and water was removed by distillation to obtain a clear liquid, determined to be mostly dialkyl ether of C₁₂-C₁₄ alcohol. The aqueous layer was evaporated to obtain a light brown mixture comprising alkyloxyethane sulfonate, sodium isethionate, and sodium di-isethionate. The amount of ethane sulfonate is about 25%.

### EXAMPLE 2

A four-necked 250 ml round-bottom flask equipped with a mechanical stirrer, an addition funnel, nitrogen inlet, and a thermocouple for temperature control is charged with 102 g (1.0 mole) C₆ alcohol and heated to about 125°-130° C. 13 g of an 80% (wt. %) aqueous solution of isethionic acid is added slowly over 10 minutes with stirring. Water from ISA is removed as it is added and the temperature is raised slowly to 160°-165° C. over 30 minutes. The ISA layer turns dark as the temperature is increased. The total mixture is neutralized with an aqueous solution comprising 50% sodium hydroxide and subsequently diluted with 50 g of water. The organic layer was subsequently separated and dried to obtain clear liquid, determined to be dialkyl ether of C₆ alcohol (60 g). The aqueous layer was evaporated to obtain a light brown mixture comprising alkyloxyethane sulfonate, sodium isethionate, and sodium di-isethionate (14 g). The amount of ethane sulfonate is about 35%.

### EXAMPLE 3

A four-necked 500 ml round-bottom flask equipped with a mechanical stirrer, an addition funnel, nitrogen inlet, and a thermocouple for temperature control is charged with 170 g (1.0 mole) C₁₀-C₁₂ alcohol and heated to about 125°-130° C. 15 g of an 80% (wt. %) aqueous solution of isethionic acid (“ISA”) is added slowly over 10 minutes with stirring. Water from ISA is removed as it is added and the temperature is raised slowly to 160°-165° C. over 30 minutes. The ISA layer turns dark as the temperature is increased but more water came over than anticipated. The total mixture is neutralized with an aqueous solution comprising 50% sodium hydroxide and subsequently diluted with 150 g of water. The organic layer is separated and dried to obtain clear liquid, determined to be dialkyl ether of C₁₀-C₁₂ alcohols (120 g). The aqueous layer was evaporated to obtain brown-colored mixture containing alkyloxyethane sulfonate, sodium isethionate, and sodium di-isethionate (23 g). The yield of ethane sulfonate is about 50%.

### EXAMPLE 4

A four-necked 1000-ml round-bottom flask equipped with a mechanical stirrer, an addition funnel, nitrogen inlet, and a thermocouple for temperature control is charged with 380 g (2.0 mole) EXXAL®12 alcohol and heated to about 125°-130° C. 16 g of an 80% (wt %) aqueous solution of isethionic acid (“ISA”) is added slowly over 10 minutes with stirring. Water from ISA is removed as it is added and the temperature is raised slowly to 160°-165° C. over 30 minutes. The reaction is continued for 14 hours. The ISA layer turns dark as the temperature is increased. The total mixture is neutralized with an aqueous solution comprising 50% sodium hydroxide and diluted with 150 g of water and the organic layer was separated and dried to obtain a clear liquid determined to be mostly dialkyl ether of EXXAL® 12 alcohol (312 g) (EXXAL is a registered trademark of Exxon Chemical Company). The aqueous layer was evaporated to obtain a brown-colored mixture of alkyloxyethane sulfonate, sodium isethionate, and sodium di-isethionate (25 g). The amount of ethane sulfonate is about 20%.

### EXAMPLE 5

A four-necked 500 ml round-bottom flask equipped with a mechanical stirrer, an addition funnel, nitrogen inlet, and a thermocouple for temperature control, is charged with 246 g (1.50 mole) C₁₀-C₁₂ alcohol and 100 grams of freshly dried sodium chloroethane sulfonate solid (0.55 moles) and stirred well while heating to 50° C. 20 grams of sodium hydroxide shot (20-40 mesh) were added over several hours in six lots and the temperature was maintained below 60° C. during the caustic addition. The temperature was raised to 120° C. and continued for one hour, and subsequently to 140° C. for 18 hours. The temperature was again raised to 160° C. for 2.5 hours and then cooled to about 100° C. The reaction mixture was then diluted with 200 g of isopropanol (“IPA”) and filtered. The filter cake was washed with more IPA and dried under vacuum at 70°-80° C. under a reduced pressure of about 30 mm. The total amount of dried solids obtained was 171 g (99% of expected). The IPA wash was concentrated and recovered about 170 g of starting material. On the basis of recovered alcohol, 94% of expected alcohol was converted to sulfonate.

### EXAMPLE 6

A four-necked 500 ml round-bottom flask equipped with a mechanical stirrer, an addition funnel, nitrogen inlet, and a thermocouple for temperature control, is charged with 750 grams (1.10 moles) of a 25% (wt. %) aqueous solution of sodium chloroethane sulfonate and stirred well while heating to 120° C. to remove water. After most of the water is removed, 400 grams of a C₁₂-C₁₄ alcohol (2 moles) was added and heating continued until the temperature had risen to 140° C. to remove all water from the reaction mixture. 44 grams of sodium hydroxide shot (20-40 mesh) were added over several hours in six lots and the temperature was maintained at 140°-145° C. during the caustic addition. The temperature was raised to 160° C., maintained for 14 hours, and then cooled to room temperature. The cooled reaction mixture is diluted with 200 g of IPA and filtered. The filter cake was washed with more IPA and then slurried with 500 g of water and treated with an effective amount (about 20 ml) of 35% (wt. %) aqueous hydrogen peroxide to effect a color change to a light yellow. Water was evaporated under nitrogen and the residue was dried under a reduced pressure of 30 mm Hg at 70°-80° C. The resulting product is 295 grams of a light brown solid which comprises ether sulfonates, and represents an 89% yield of the theoretical basis.

### EXAMPLE 7

A four-necked 500 ml round-bottom flask equipped with a mechanical stirrer, an addition funnel, nitrogen inlet, and a thermocouple for temperature control, is charged with 103 grams (1.30 moles) of a 50% (wt. %) aqueous sodium hydroxide solution, 211 grams (1.05 moles) of a C₁₂-C₁₄ alcohol mixture while stirring well and heating to 140° C. to remove water. After most of the water is removed, 108 grams (0.60 moles) of 77% chloroethane sulfonic acid was added slowly over 2 hours. After the addition, the temperature was raised to 160° C. and stirring was continued for 14 hours. The cooled reaction mixture is diluted with 200 g of EPA and filtered. The filter cake was washed with more IPA and the solids were dried at 70°-80° C. under a reduced pressure of 30 mm. The product ether sulfonates appeared as a light yellow solid, and weighed 145 g (82% of theoretical).

### EXAMPLE 8

A four-necked 500 ml round-bottom flask equipped with a mechanical stirrer, an addition funnel, nitrogen inlet, and a thermocouple for temperature control, is charged with 148 g (0.50 mole) SURFONIC® L12-3 ethoxylated surfactant and heated to about 50°-55° C. 125 grams (0.67 moles) of 90% sodium chloroethane sulfonate solid is added and stirred well. 30 grams of sodium hydroxide shot (20-40 mesh) were then added over several hours in six lots while maintaining the temperature below 60° C. during the caustic addition. The temperature of the flask contents were maintained at 60°-65° C. for 24 hours. The reaction mixture turned slightly yellow and was diluted with 200 g of water, transferred into a tared bottle, and determined to comprise 450 g of reaction product. A sample was analyzed for sulfonate and found to contain about 17%, which yields approximately 50% of ether sulfonate.

### EXAMPLE 9

A four-necked 500 ml Round-bottom flask equipped with a mechanical stirrer, an addition funnel, nitrogen inlet, and a thermocouple for temperature control is charged with 142.5 grams (0.50 moles) of SURFONIC® L24-2 ethoxylated surfactant and heated to about 75°-80° C. 130 grams of a 25% (wt. %) solution of sodium methoxide in methanol (0.60 moles NaOEt ) was added slowly over 30 minutes and methanol was flashed off. After addition of all of the methoxide solution, 90 gams of 90% sodium chloroethane sulfonate (0.50 moles) was added to the flask in three lots at 30 minute intervals and stirring was continued for 20 hours, while maintaining the contents of the flask at 75° C. The heating was then stopped, and the contents of the flask were cooled to 50° C. About 200 g of 15% H₂SO₄ was then slowly added to neutralize the sulfonate salt. The mixture became a white paste, and the hot paste was transferred into a tared bottle and determined to contain 425 g of material comprising about 40% ether sulfonates.

### EXAMPLE 10

A four-necked 1000 ml round-bottom flask equipped with a mechanical stirrer, an addition funnel, nitrogen inlet, and a thermocouple for temperature control is charged with 285 grams (1.00 moles) SURFONIC® L24-2 ethoxylated alcohol. 92.5 grams (0.50 moles) of 90% chloroethane sulfonate is added, and the flask heated until a temperature of about 75°-80° C. is reached. 20 grams of 20-40 mesh sodium hydroxide shot (0.50 moles) were added in six lots over the course of about 2 hours, and the temperature was allowed rise to about 120° C. The mixture remained fluid and the reaction temperature was raised to and maintained at 140° C. for 12 hours. The heating was then stopped, and the flask contents cooled to about 100° C. About 300 g of IPA was added to dissolve unreacted SURFONIC® L24-2 ethoxylated alcohol. The sulfonate salt was filtered and dried at 80° C. under a reduced pressure of about 30 mm Hg. The light brown soft solid product was transferred into a tared bottle which was determined to contain 187 grams of material that comprises 74% ether sulfonates.

### EXAMPLE 11

A four-necked 500 ml round-bottom flask equipped with a mechanical stirrer, an addition funnel, nitrogen inlet, and a thermocouple for temperature control is charged with 487 grams (1.0 moles) SURFONIC® L24-7 ethoxylated alcohol and heated to about 75°-85° C. 130 grams of a 25% solution of sodium methoxide in methanol (0.60 moles) was added slowly over 2 hours, at which time the methanol was flashed off. After all methanol is removed, 90 grams of 90% (wt. %) sodium chloroethane sulfonate (0.60 moles) was added in three lots at 30 minutes intervals, and stirring continued for about 20 hrs at 60° C. Next, the temperature of the flask contents was raised to and maintained at 70° C. for another 24 hours. The off-white product material was transferred into a tared bottle which was determined to contain 610 g of material having an active sulfonate content of 45%, which was about 84% of the theoretical value.

### EXAMPLE 12

A four-necked 500 ml round-bottom flask equipped with a mechanical stirrer, an addition funnel, nitrogen inlet, and a thermocouple for temperature control is charged with 110 grams (0.50 moles) of mono-nonyl phenol and heated to about 75°-85° C. 22 grams (0.55 moles) of solid 90% sodium chloroethane sulfonate was added, and the contents of the flask were stirred well. To the thick mass were added 22 grams (0.55 moles) of 20-40 mesh sodium hydroxide shot over the course of about 2 hours. During the addition the temperature of the flask contents rose to 90° C. The reaction mixture became difficult to mix, and was diluted with 112 g of diglyme, and stirring at 90° C. continued for 2 hours. The mixture was then neutralized with 200 grams of 15% sulfuric acid and subsequently heated to dissolve all the solids. The organic layer was allowed to separate and was isolated using a separatory funnel. The separated organic layer comprised about 256 g of cloudy material, which was heated under a reduced pressure of about 30 mm Hg at 130° C. to remove diglyme. The resulting residue comprised a total of 180 g of light brown pasty material, and contained about 31% of desired MNP ether sulfonate.

### EXAMPLE 13

A four-necked 1000 ml round-bottom flask equipped with a mechanical stirrer, an addition funnel, nitrogen inlet, and a thermocouple for temperature control is charged with 175 grams (0.25 moles) SURFONIC® L24-12 ethoxylated alcohol and 50 grams (0.25 moles) of 84% (wt. %) solid sodium chloroethane sulfonate. The contents of the flask are heated to about 120°-125° C., and then 10 grams (0.25 moles) of 20-40 mesh sodium hydroxide shot were added in six lots over the course of about 2 hours. The reaction temperature is raised to 140° C. and maintained at 140 ° C. for 23 hours with stirring. The light brown mixture that resulted was cooled to 100° C., poured into a tared bottle, and determined to contain 46.50% active surfactants.

Consideration must be given to the fact that although this invention has been described and disclosed in relation to certain preferred embodiments, obvious equivalent modifications and alterations thereof will become apparent to one of ordinary skill in this art upon reading and understanding this specification and the claims appended hereto. Accordingly, the presently disclosed invention is intended to cover all such modifications and alterations, and is limited only by the scope of the claims which follow.

What is claimed is:

1) A process for producing an alcohol ether sulfonate by reacting an alcohol with isethionic acid according to the reaction: 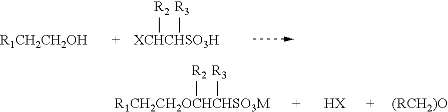
in which:
R₁ is independently any straight-chain, branched, or cyclic, saturated or unsaturated, hydrocarbyl moiety that is selected from the group consisting of: 1) any C₅-C₁₉ alkyl group; 2) any C₅-C₁₉ aryl group; 3) any C₅-C₁₉ alkylaryl group; 4) any R₄(CH₂CH₂O)ₙ— group, in which R₄ is any C₃-C₂₄ alkyl, aryl, or alkylaryl group, whether straight-chain, branched, or cyclic, saturated or unsaturated, and in which n independently has any value between about 2 and 25;
R₂ and R₃ are each independently selected from the group consisting of: hydrogen, methyl and ethyl;
X is selected from the group consisting of: chlorine, bromine, or hydroxy; and
M is selected from the group consisting of: Na, K, Li, Ca, Mg, and hydrogen, by contacting the isethionic acid or its halo-derivative and the primary alcohol at any temperature in the range of about 60° C. to about 200° C., and at any pressure in the range of between about 50 and 760 mm Hg.

2) A composition of matter useful for cleaning hard surfaces, laundry, and the human body comprising:
a) a first component which comprises an anionic form of the alcohol ether sulfonate described by the formula: 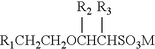
in which R₁ is independently any straight-chain, branched, or cyclic, saturated or unsaturated, hydrocarbyl moiety that is selected from the group consisting of: 1) any C₅-C₁₉ alkyl group; 2) any C₅-C₁₉ aryl group; 3) any C₅-C₁₉ alkylaryl group; 4) any R₄(CH₂CH₂O)ₙ— group, in which R₄ is any C₃-C₂₄ alkyl, aryl, or alkylaryl group, whether straight-chain, branched, or cyclic, saturated or unsaturated, and in which n independently has any value between about 2 and 25; R₂ and R₃ are each independently selected from the group consisting of: hydrogen, methyl and ethyl; and M is selected from the group consisting of: Na, K, Li, Ca, Mg, and hydrogen; and
b) a second component selected from the group consisting of: fatty acids, alkyl sulfates, ethanolamines, amine oxides, alkali carbonates, water, ethanol, isopropanol, pine oil, sodium chloride, sodium silicate, polymers, alcohol alkoxylates, zeolites, perborate salts, alkali sulfates, enzymes, hydrotropes, dyes, fragrances, preservatives, brighteners, builders, polyacrylates, essential oils, alkali hydroxides, ether sulfates, alkylphenol ethoxylates, fatty acid amides, alpha olefin sulfonates, paraffin sulfonates, betaines, chelating agents, tallowamine ethoxylates, polyetheramine ethoxylates, ethylene oxide/propylene oxide block copolymers, alcohol ethylene oxide/propylene oxide low foam surfactants, methyl ester sulfonates, alkyl polysaccharides, N-methyl glucamides, alkylated sulfonated diphenyl oxide, and water soluble alkylbenzene sulfonates or alkyltoluene sulfonates, regardless of their 2-phenyl isomer content or degree of branching or linearity in the alkyl chain.
